(19) United States
(12) Patent Application Publication
(10) Pub. No.: US 2023/0312578 A1
(43) Pub. Date: Oct. 5, 2023

(54) 6H-INDOLO[2,3-B]QUINOXALINE DERIVATIVES USEFUL IN THERAPY

(71) Applicant: Vironova Medical AB, Stockholm (SE)

(72) Inventors: Jan BERGMAN, Spånga (SE); Ngarita KINGI, Hägersten (SE); Eva O'KEEFFE, Stockholm (SE); Mohammed HOMMAN, Nacka (SE)

(21) Appl. No.: 18/003,750

(22) PCT Filed: Jun. 29, 2021
(86) PCT No.: PCT/EP2021/067786

§ 371 Date: Dec. 29, 2022

(30) Foreign Application Priority Data
Jun. 29, 2020 (EP) 20182909.0
Oct. 15, 2020 (EP) 20202078.0
Dec. 22, 2020 (EP) 20216659.1

(51) Int. Cl. C07D 487/04; A61P 35/00

(52) U.S. Cl. CPC C07D 487/04; A61P 35/00

(57) ABSTRACT

A compound of formula (I) or a pharmaceutically acceptable salt thereof, useful in therapy. A pharmaceutical composition comprising a compound of formula (I). The compound is useful in the treatment of cancer.

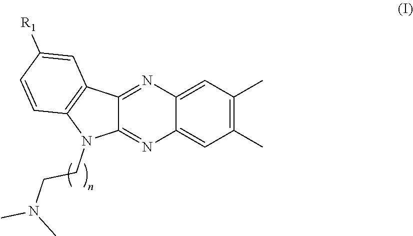

### FIELD OF THE INVENTION

The present invention relates to certain 6H-indolo[2,3-b]quinoxaline derivatives for use in therapy, in particular in the treatment of cancer.

### BACKGROUND OF THE INVENTION

Various 6H-indolo[2,3-b]quinoxaline derivatives have been previously disclosed as pharmaceutically useful. For example, some substituted indoloquinoxalines are disclosed in international application No. PCT/SE87/00019 (published as WO 87/04436) as having antiviral effect and an anticancer effect.

International application No. PCT/SE95/01581 (published as WO 96/19996) discloses some further substituted indoloquinoxalines as having a DNA protecting effect in the initial phase and/or the promoting phase of carcinogenesis and as capable of preventing oxidative stress in patients with diseases related to free radicals. In particular, the compound B220 (2-(2,3-dimethyl-6H-indolo[2,3-b]quinoxalin-6-yl)-N,N-dimethylethanamine) and its 9-hydroxy derivative (6-(2-(dimethylamino)ethyl)-2,3-dimethyl-6H-indolo[2,3-b]quinoxalin-9-ol) were mentioned and claimed as therapeutically active compounds, and B-220 and its 9-hydroxy derivative were indicated to have similar anti-mutagenic potential, without any particular cancer being mentioned.

To prepare the 9-hydroxylated derivative, WO 96/19996 teaches a multi-step process comprising an initial step of nitration in 9-position to obtain the corresponding 9-nitro compound as a synthetic intermediate, the hydrogenation of which leads to the corresponding 9-amino compound, which subsequently is submitted to a diazotization, followed by treatment with a Cu-based catalyst to give the desired 9-hydroxy compound. WO 96/19996 does not suggest that the 9-nitro compound or the 9-amino compound could have any therapeutic activity.

In spite of the development of new anti-cancer drugs, there remains a need for novel drugs for the treatment of cancer, preferably with reduced side effects, such as toxicity effects.

### SUMMARY OF THE INVENTION

In one aspect, therapeutically useful compounds are provided, compounds having a favourable therapeutic window, in terms of high therapeutic activity compared to toxic side effects.

Compounds having favourable characteristics are certain 2-(2,3-dimethyl-6H-indolo[2,3-b]quinoxalin-6-yl)-N,N-dimethylethanamine derivatives having an electron withdrawing moiety attached in 9-position.

An aspect, therefore, is a compound of formula (I)

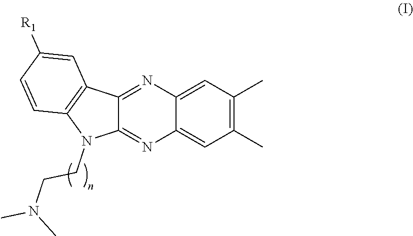

or a pharmaceutically acceptable salt thereof, wherein
n is 1 or 2; and
R₁ is a moiety selected from NO₂, NH₂, CN, NH₃⁺, and CF₃O,
for use in therapy.

A further aspect is a pharmaceutical composition comprising a compound of formula (I) as defined herein above, or pharmaceutically acceptable salt thereof, and optionally a pharmaceutically acceptable excipient.

A further aspect is a compound of formula (I) as defined herein above, or pharmaceutically acceptable salt thereof, for use in therapy, wherein the compound or pharmaceutically acceptable salt thereof is administered in combination with a further therapeutic agent.

A further aspect is a compound of formula (I) as defined herein above, or a pharmaceutically acceptable salt thereof, for use in the treatment of cancer, e.g. cancer selected from a hematological cancer, lung cancer, colon cancer, cancer of the CNS, skin cancer, ovarian cancer, renal cancer, prostate cancer and breast cancer.

Also provided herein is a compound of formula (I)

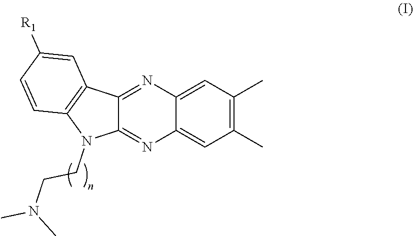

or a pharmaceutically acceptable salt thereof, wherein
n is 1 or 2; and
R₁ is a moiety selected from OH, CH₃O, NO₂, NH₂, CN, CF₃, NH₃⁺, and CF₃O,
for use in the treatment of a cancer selected from a hematological cancer, lung cancer, colon cancer, cancer of the CNS, skin cancer, ovarian cancer, renal cancer, prostate cancer and breast cancer.

A further aspect is a compound of formula (I) as defined herein above, or pharmaceutically acceptable salt thereof, for use in the treatment of cancer, wherein the compound or pharmaceutically acceptable salt thereof is administered in combination with a further therapeutic agent.

A further aspect is the use of a compound of formula (I)

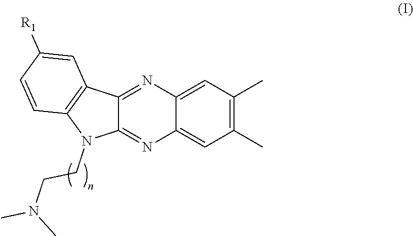

or a pharmaceutically acceptable salt thereof, wherein
n is 1 or 2; and
R₁ is selected from OH, NO₂, NH₂, CN, CF₃, NH₃⁺, CH₃O, and CF₃O,
in the manufacture of a medicament for the treatment a cancer selected from a hematological cancer, lung cancer, colon cancer, cancer of the CNS, skin cancer, ovarian cancer, renal cancer, prostate cancer, and breast cancer.

A further aspect is a method for the treatment of a cancer selected from a hematological cancer, lung cancer, colon cancer, cancer of the CNS, skin cancer, ovarian cancer, renal cancer, prostate cancer and breast cancer in a mammal by administering, to a mammal in need of such treatment, an effective amount of a compound of formula (I)

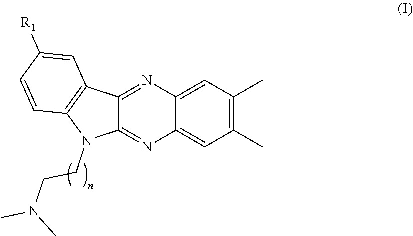

or a pharmaceutically acceptable salt thereof, wherein
n is 1 or 2; and
R₁ is selected from OH, NO₂, NH₂, CN, CF₃, NH₃⁺, CH₃O, and CF₃O.

A further aspect is a compound of formula (I)

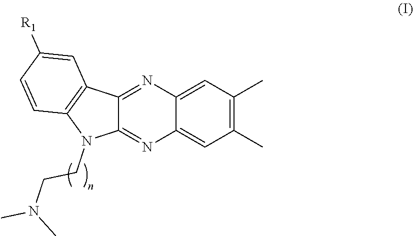

or a pharmaceutically acceptable salt thereof, wherein n is 1 or 2; and R₁ is CN.

Further aspects and embodiments are as described herein below.

### BRIEF DESCRIPTION OF THE DRAWING

FIG. 1 shows a semilogarithmic plot of the growth inhibition of leukemia cells line K-562, in the presence of different concentrations of 2-(2,3-dimethyl-6H-indolo[2,3-b]quinoxalin-6-yl)-N,N-dimethylethanamine (B220, not according to the invention), 2-(2,3-dimethyl-9-nitro-6H-indolo[2,3-b]quinoxalin-6-yl)-N,N-dimethylethanamine (JBA-185), 6-(2-(dimethylamino)ethyl)-2,3-dimethyl-6H-indolo[2,3-b]quinoxalin-9-amine (JBD-136), 6-(2-(dimethylamino)ethyl)-2,3-dimethyl-6H-indolo[2,3-b]quinoxalin-9-ol (JBD-139), and 2,3-dimethyl-6-(N,N-dimethylaminoethyl)-9-methoxy-6H-indolo-(2,3-b)quinoxaline (JBA-094), respectively. The y-axis shows growth, where 100% means no growth inhibition, 0 is total growth inhibition, and −50 means 50% lethality. The x-axis shows the 10-logarithm of the compound concentration (in M).

FIG. 2 shows semilogarithmic plots of the growth inhibition of prostate cancer cell line DU-145 in the presence of different concentrations of 2-(2,3-dimethyl-6H-indolo[2,3-b]quinoxalin-6-yl)-N,N-dimethylethanamine (B220, not according to the invention), 2-(2,3-dimethyl-9-nitro-6H-indolo[2,3-b]quinoxalin-6-yl)-N,N-dimethylethanamine (JBA-185), 6-(2-(dimethylamino)ethyl)-2,3-dimethyl-6H-indolo[2,3-b]quinoxalin-9-amine (JBD-136), 6-(2-(dimethylamino)ethyl)-2,3-dimethyl-6H-indolo[2,3-b]quinoxalin-9-ol (JBD-139), and 2,3-dimethyl-6-(N,N-dimethylaminoethyl)-9-methoxy-6H-indolo-(2,3-b)quinoxaline (JBA-094), respectively. The y-axis shows growth, where 100% means no growth inhibition, 0 is total growth inhibition, and −50 means 50% lethality. The x-axis shows the 10-logarithm of the compound concentration (in M).

FIG. 3 shows semilogarithmic plots of the growth inhibition of ovarian cancer cell line OVCAR-3 in the presence of different concentrations of 2-(2,3-dimethyl-6H-indolo[2,3-b]quinoxalin-6-yl)-N,N-dimethylethanamine (B220, not according to the invention), 2-(2,3-dimethyl-9-nitro-6H-indolo[2,3-b]quinoxalin-6-yl)-N,N-dimethylethanamine (JBA-185), 6-(2-(dimethylamino)ethyl)-2,3-dimethyl-6H-indolo[2,3-b]quinoxalin-9-amine (JBD-136), 6-(2-(dimethylamino)ethyl)-2,3-dimethyl-6H-indolo[2,3-b]quinoxalin-9-ol (JBD-139), and 2,3-dimethyl-6-(N,N-dimethylaminoethyl)-9-methoxy-6H-indolo-(2,3-b)quinoxaline (JBA-094), respectively. The y-axis shows growth, where 100% means no growth inhibition, 0 is total growth inhibition, and −50 means 50% lethality. The x-axis shows the 10-logarithm of the compound concentration (in M).

FIG. 4 shows semilogarithmic plots of the growth inhibition of non-small cell lung cancer (NSCLC) cell line NCI-H322M in the presence of different concentrations of 2-(2,3-dimethyl-6H-indolo[2,3-b]quinoxalin-6-yl)-N,N-dimethylethanamine (B220, not according to the invention), 2-(2,3-dimethyl-9-nitro-6H-indolo[2,3-b]quinoxalin-6-yl)-N,N-dimethylethanamine (JBA-185), 6-(2-(dimethylamino)ethyl)-2,3-dimethyl-6H-indolo[2,3-b]quinoxalin-9-amine (JBD-136), 6-(2-(dimethylamino)ethyl)-2,3-dimethyl-6H-indolo[2,3-b]quinoxalin-9-ol (JBD-139), and 2,3-dimethyl-6-(N,N-dimethylaminoethyl)-9-methoxy-6H-indolo-(2,3-b)quinoxaline (JBA-094), respectively. The y-axis shows growth, where 100% means no growth inhibition, 0 is total growth inhibition, and −50 means 50% lethality. The x-axis shows the 10-logarithm of the compound concentration (in M).

### DETAILED DESCRIPTION

Unless otherwise specified, any term used herein is to be given its conventional meaning.

The term OH (or hydroxy) refers to a moiety of formula

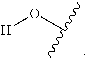 [SMILES: *O]

The term CH₃O (or methoxy) refers to a moiety of formula

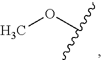 [SMILES: *OC]

which moiety may also be represented as

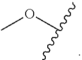 [SMILES: *OC]

The term NO₂ (or nitro) refers to a moiety of formula

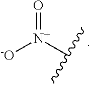

The term NH₂ (or amino) refers to a moiety of formula

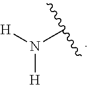 [SMILES: *N]

The term CN (or cyano) refers to a moiety of formula

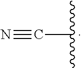 [SMILES: *C#N]

The term CF₃ (or trifluoromethyl) refers to a moiety of formula

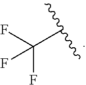 [SMILES: *C(F)(F)F]

The term NH₃⁺ refers to a moiety of formula

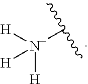 [SMILES: *[NH3+]]

The term CF₃O (or trifluoromethoxy) refers to a moiety of formula

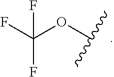 [SMILES: *OC(F)(F)F]

“Optional” or “optionally” means that the subsequently described event or circumstance may but need not occur, and that the description includes instances where the event or circumstance occurs and instances in which it does not.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

The term “excipient” refers to a pharmaceutically acceptable chemical, such as known to those of ordinary skill in the art of pharmacy to aid in the administration of the medicinal agent. It is a compound that is useful in preparing a pharmaceutical composition, generally safe, non-toxic and neither biologically nor otherwise undesirable, and includes excipients that are acceptable for veterinary use as well as human pharmaceutical use. Exemplary excipients include binders, surfactants, diluents, disintegrants, antiadherents, and lubricants.

“Therapeutically effective amount” means an amount of a compound that, when administered to a subject for treating a disease state, is sufficient to effect such treatment for the disease state. The “therapeutically effective amount” will vary depending on the compound, the disease state being treated, the severity of the disease treated, the age and relative health of the subject, the route and form of administration, the judgment of the attending medical or veterinary practitioner, etc.

As used herein the terms “treatment” or “treating” is an approach for obtaining beneficial or desired results including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total) whether detectable or undetectable.

The term can also mean prolonging survival as compared to expected survival without the treatment.

The term “mammal” refers to a human or any mammalian animal, e.g. a primate, a farm animal, a pet animal, or a laboratory animal. Examples of such animals are monkeys, cows, sheep, horses, pigs, dogs, cats, rabbits, mice, rats etc. Preferably, the mammal is a human.

The term “cancer” refers to any malignant cell growth or tumor in the body of a mammal, caused by abnormal and uncontrolled cell division; it may spread to other parts of the body through the lymphatic system or the blood stream and includes both solid tumors and bloodborne tumors.

Exemplary cancers include adrenocortical carcinoma, AIDS-related cancers, AIDS-related lymphoma, anal cancer, anorectal cancer, appendix cancer, childhood cerebellar astrocytoma, childhood cerebral astrocytoma, basal cell carcinoma, biliary cancer, extrahepatic bile duct cancer, intrahepatic bile duct cancer, urinary bladder cancer, bone and joint cancer, osteosarcoma and malignant fibrous histiocytoma, brain tumor, brain stem glioma, cerebellar astrocytoma, cerebral astrocytoma/malignant glioma, ependymoma, medulloblastoma, visual pathway and hypothalamic glioma, breast cancer, bronchial adenomas/carcinoids, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Sezary syndrome, endometrial cancer, esophageal cancer, extracranial germ cell tumor, extragonadal germ cell tumor, eye cancer, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin's lymphoma, hypopharyngeal cancer, ocular cancer, Kaposi's sarcoma, renal cancer, laryngeal cancer, acute lymphoblastic leukemia, acute myeloid leukemia, hairy cell leukemia, lip and oral cavity cancer, lung cancer, non-small cell lung cancer, small cell lung cancer, non-Hodgkin's lymphoma, primary central nervous system lymphoma, Waldenstrom's macroglobulinemia, intraocular (eye) melanoma, Merkel cell carcinoma, malignant mesothelioma, metastatic squamous neck cancer, cancer of the tongue, multiple endocrine neoplasia syndrome, myelodysplastic syndromes, myelodysplastic/myeloproliferative diseases, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pheochromocytoma, pineoblastoma and supratentorial primitive neuroectodermal tumors, pituitary tumor, plasma cell neoplasm/multiple myeloma, pleuropulmonary blastoma, prostate cancer, rhabdomyosarcoma, salivary gland cancer, Ewing's sarcoma family of tumors, soft tissue sarcoma, uterine cancer, uterine sarcoma, skin cancer (non-melanoma), skin cancer (melanoma), small intestine cancer, squamous cell carcinoma, testicular cancer, throat cancer, thymoma, thymoma and thymic carcinoma, thyroid cancer, transitional cell cancer of the renal pelvis and ureter and other urinary organs, gestational trophoblastic tumor, urethral cancer, vaginal cancer, vulvar cancer, and Wilm's tumor.

In the compound of formula (I)

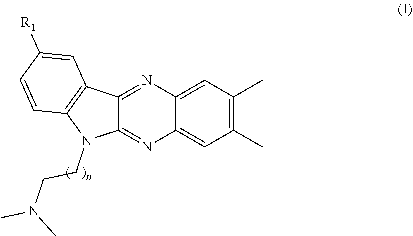

for use as defined herein, n is an integer 1 or 2.

In some embodiments, n is 2, i.e. the dimethylamino moiety is linked to the polycyclic ring system by propylene chain: —(CH₂)₃—.

In some other embodiments, n is 1, i.e. the compound of formula (I) is a compound of formula (Ia)

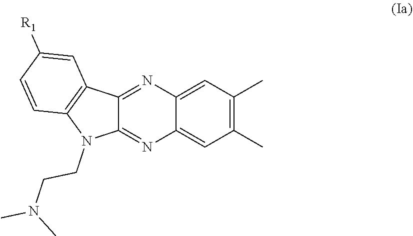

wherein R₁ is as defined herein.

In a compound of formula (I) for use in the treatment of cancer, e.g. cancer selected from a hematological cancer, lung cancer, colon cancer, cancer of the CNS, skin cancer, ovarian cancer, renal cancer, prostate cancer and breast cancer, the moiety R₁ may be selected from OH, NO₂, NH₂, CN, CF₃, NH₃₄, CH₃O, and CF₃O.

In some embodiments, R₁ is selected from OH, NO₂, NH₂, CN, CF₃, NH₃⁺, and CF₃O.

In some embodiments, R₁ is selected from NO₂, NH₂, CN, CF₃, NH₃⁺, and CF₃O.

In some embodiments, R₁ is selected from NO₂, CN, CF₃, NH₃⁺, and CF₃O.

In some embodiments, R₁ is selected from NO₂, NH₂, CF₃, NH₃⁺, and CF₃O.

In some embodiments, R₁ is selected from NO₂, NH₂, CN, NH₃⁺, and CF₃O.

In some embodiments, R₁ is selected from NO₂, NH₂, CN, CF₃, and CF₃O.

In some embodiments, R₁ is selected from NO₂, NH₂, CN, CF₃, and NH₃⁺.

In some embodiments, R₁ is selected from NO₂, CF₃, NH₃⁺, and CF₃O.

In some embodiments, R₁ is selected from NO₂, NH₂, NH₃⁺, and CF₃O.

In some embodiments, R₁ is selected from NO₂, NH₂, CN, and CF₃O.

In some embodiments, R₁ is selected from NO₂, NH₂, CN, and NH₃⁺.

In some embodiments, R₁ is selected from NO₂, NH₂, CN, and CF₃.

In some embodiments, R₁ is selected from NO₂, CN, CF₃, and CF₃O.

In some embodiments, R₁ is selected from NO₂, CN, NH₃⁺, and CF₃O.

In some embodiments, R₁ is selected from NO₂, CN, CF₃, and NH₃⁺.

In some embodiments, R₁ is selected from NO₂, NH₂, CF₃, and NH₃₄.

In some embodiments, R₁ is selected from NO₂, NH₂, CF₃, and CF₃O.

In some embodiments, R₁ is selected from NO₂, NH₃⁺, and CF₃O.

In some embodiments, R₁ is selected from NO₂, CF₃, and CF₃O.

In some embodiments, R₁ is selected from NO₂, NH₂, and CF₃O.

In some embodiments, R₁ is selected from NO₂, NH₂, and NH₃⁺.

In some embodiments, R₁ is selected from NO₂, NH₂, and CN.

In some embodiments, R₁ is selected from NO₂, CN, and CF₃.

In some embodiments, R₁ is selected from NO₂, CF₃, and NH₃⁺.

In some embodiments, R₁ is selected from NO₂, CN, and CF₃O.

In some embodiments, R₁ is selected from NO₂, NH₂, and CF₃.

In some embodiments, R₁ is selected from NO₂, CN, and NH₃⁺.

In some embodiments, R₁ is selected from NO₂, NH₂, and CN.

In some embodiments, R₁ is selected from NO₂ and CF₃O.

In some embodiments, R₁ is selected from NO₂ and NH₃⁺.

In some embodiments, R₁ is selected from NO₂ and CF₃.

In some embodiments, R₁ is selected from NO₂ and CN.

In some embodiments, R₁ is selected from NO₂ and NH₂.

In some embodiments, R₁ is selected from OH, NO₂, CN, CF₃, NH₃⁺, and CF₃O.

In some embodiments, R₁ is selected from OH, NO₂, NH₂, CF₃, NH₃⁺, and CF₃O.

In some embodiments, R₁ is selected from OH, NO₂, NH₂, CN, NH₃⁺, and CF₃O.

In some embodiments, R₁ is selected from OH, NO₂, NH₂, CN, CF₃, and CF₃O.

In some embodiments, R₁ is selected from OH, NO₂, NH₂, CN, CF₃, and NH₃⁺.

In some embodiments, R₁ is selected from OH, NO₂, CF₃, NH₃⁺, and CF₃O.

In some embodiments, R₁ is selected from OH, NO₂, NH₂, NH₃⁺, and CF₃O.

In some embodiments, R₁ is selected from OH, NO₂, NH₂, CN, and CF₃O.

In some embodiments, R₁ is selected from OH, NO₂, NH₂, CN, and NH₃⁺.

In some embodiments, R₁ is selected from OH, NO₂, NH₂, CN, and CF₃.

In some embodiments, R₁ is selected from OH, NO₂, CN, CF₃, and CF₃O.

In some embodiments, R₁ is selected from OH, NO₂, CN, NH₃⁺, and CF₃O.

In some embodiments, R₁ is selected from OH, NO₂, CN, CF₃, and NH₃⁺.

In some embodiments, R₁ is selected from OH, NO₂, NH₂, CF₃, and NH₃⁺.

In some embodiments, R₁ is selected from OH, NO₂, NH₂, CF₃, and CF₃O.

In some embodiments, R₁ is selected from OH, NO₂, NH₃⁺, and CF₃O.

In some embodiments, R₁ is selected from OH, NO₂, CF₃, and CF₃O.

In some embodiments, R₁ is selected from OH, NO₂, NH₂, and CF₃O.

In some embodiments, R₁ is selected from OH, NO₂, NH₂, and NH₃₄.

In some embodiments, R₁ is selected from OH, NO₂, NH₂, and CN.

In some embodiments, R₁ is selected from OH, NO₂, CN, and CF₃.

In some embodiments, R₁ is selected from OH, NO₂, CF₃, and NH₃₄.

In some embodiments, R₁ is selected from OH, NO₂, CN, and CF₃O.

In some embodiments, R₁ is selected from OH, NO₂, NH₂, and CF₃.

In some embodiments, R₁ is selected from OH, NO₂, CN, and NH₃⁺.

In some embodiments, R₁ is selected from OH, NO₂, NH₂, and CN.

In some embodiments, R₁ is selected from OH, NO₂ and CF₃O.

In some embodiments, R₁ is selected from OH, NO₂ and NH₃⁺.

In some embodiments, R₁ is selected from OH, NO₂ and CF₃.

In some embodiments, R₁ is selected from OH, NO₂ and CN.

In some embodiments, R₁ is selected from OH, NO₂ and NH₂.

In some embodiments, R₁ is selected from OH and NO₂.

In some embodiments, R₁ is selected from CH₃O, NO₂, NH₂, CN, CF₃, NH₃⁺, and CF₃O.

In some embodiments, R₁ is selected from CH₃O, NO₂, CN, CF₃, NH₃₄, and CF₃O.

In some embodiments, R₁ is selected from CH₃O, NO₂, NH₂, CF₃, NH₃⁺, and CF₃O.

In some embodiments, R₁ is selected from CH₃O, NO₂, NH₂, CN, NH₃₄, and CF₃O.

In some embodiments, R₁ is selected from CH₃O, NO₂, NH₂, CN, CF₃, and CF₃O.

In some embodiments, R₁ is selected from CH₃O, NO₂, NH₂, CN, CF₃, and NH₃⁺.

In some embodiments, R₁ is selected from CH₃O, NO₂, CF₃, NH₃⁺, and CF₃O.

In some embodiments, R₁ is selected from CH₃O, NO₂, NH₂, NH₃⁺, and CF₃O.

In some embodiments, R₁ is selected from CH₃O, NO₂, NH₂, CN, and CF₃O.

In some embodiments, R₁ is selected from CH₃O, NO₂, NH₂, CN, and NH₃⁺.

In some embodiments, R₁ is selected from CH₃O, NO₂, NH₂, CN, and CF₃.

In some embodiments, R₁ is selected from CH₃O, NO₂, CN, CF₃, and CF₃O.

In some embodiments, R₁ is selected from CH₃O, NO₂, CN, NH₃⁺, and CF₃O.

In some embodiments, R₁ is selected from CH₃O, NO₂, CN, CF₃, and NH₃₄.

In some embodiments, R₁ is selected from CH₃O, NO₂, NH₂, CF₃, and NH₃⁺.

In some embodiments, R₁ is selected from CH₃O, NO₂, NH₂, CF₃, and CF₃O.

In some embodiments, R₁ is selected from CH₃O, NO₂, NH₃⁺, and CF₃O.

In some embodiments, R₁ is selected from CH₃O, NO₂, CF₃, and CF₃O.

In some embodiments, R₁ is selected from CH₃O, NO₂, NH₂, and CF₃O.

In some embodiments, R₁ is selected from CH₃O, NO₂, NH₂, and NH₃⁺.

In some embodiments, R₁ is selected from CH₃O, NO₂, NH₂, and CN.

In some embodiments, R₁ is selected from CH₃O, NO₂, CN, and CF₃.

In some embodiments, R₁ is selected from CH₃O, NO₂, CF₃, and NH₃⁺.

In some embodiments, R₁ is selected from CH₃O, NO₂, CN, and CF₃O.

In some embodiments, R₁ is selected from CH₃O, NO₂, NH₂, and CF₃.

In some embodiments, R₁ is selected from CH₃O, NO₂, CN, and NH₃⁺.

In some embodiments, R₁ is selected from CH₃O, NO₂, NH₂, and CN.

In some embodiments, R₁ is selected from CH₃O, NO₂ and CF₃O.

In some embodiments, R₁ is selected from CH₃O, NO₂ and NH₃⁺.

In some embodiments, R₁ is selected from CH₃O, NO₂ and CF₃.

In some embodiments, R₁ is selected from CH₃O, NO₂ and CN.

In some embodiments, R₁ is selected from CH₃O, NO₂ and NH₂.

In some embodiments, R₁ is selected from OH, CH₃O, NO₂, CN, CF₃, NH₃⁺, and CF₃O.

In some embodiments, R₁ is selected from OH, CH₃O, NO₂, NH₂, CF₃, NH₃⁺, and CF₃O.

In some embodiments, R₁ is selected from OH, CH₃O, NO₂, NH₂, CN, NH₃⁺, and CF₃O.

In some embodiments, R₁ is selected from OH, CH₃O, NO₂, NH₂, CN, CF₃, and CF₃O.

In some embodiments, R₁ is selected from OH, CH₃O, NO₂, NH₂, CN, CF₃, and NH₃⁺.

In some embodiments, R₁ is selected from OH, CH₃O, NO₂, CF₃, NH₃⁺, and CF₃O.

In some embodiments, R₁ is selected from OH, CH₃O, NO₂, NH₂, NH₃⁺, and CF₃O.

In some embodiments, R₁ is selected from OH, CH₃O, NO₂, NH₂, CN, and CF₃O.

In some embodiments, R₁ is selected from OH, CH₃O, NO₂, NH₂, CN, and NH₃⁺.

In some embodiments, R₁ is selected from OH, CH₃O, NO₂, NH₂, CN, and CF₃.

In some embodiments, R₁ is selected from OH, CH₃O, NO₂, CN, CF₃, and CF₃O.

In some embodiments, R₁ is selected from OH, CH₃O, NO₂, CN, NH₃⁺, and CF₃O.

In some embodiments, R₁ is selected from OH, CH₃O, NO₂, CN, CF₃, and NH₃⁺.

In some embodiments, R₁ is selected from OH, CH₃O, NO₂, NH₂, CF₃, and NH₃⁺.

In some embodiments, R₁ is selected from OH, CH₃O, NO₂, NH₂, CF₃, and CF₃O.

In some embodiments, R₁ is selected from OH, CH₃O, NO₂, NH₃⁺, and CF₃O.

In some embodiments, R₁ is selected from OH, CH₃O, NO₂, CF₃, and CF₃O.

In some embodiments, R₁ is selected from OH, CH₃O, NO₂, NH₂, and CF₃O.

In some embodiments, R₁ is selected from OH, CH₃O, NO₂, NH₂, and NH₃⁺.

In some embodiments, R₁ is selected from OH, CH₃O, NO₂, NH₂, and CN.

In some embodiments, R₁ is selected from OH, CH₃O, NO₂, CN, and CF₃.

In some embodiments, R₁ is selected from OH, CH₃O, NO₂, CF₃, and NH₃⁺.

In some embodiments, R₁ is selected from OH, CH₃O, NO₂, CN, and CF₃O.

In some embodiments, R₁ is selected from OH, CH₃O, NO₂, NH₂, and CF₃.

In some embodiments, R₁ is selected from OH, CH₃O, NO₂, CN, and NH₃⁺.

In some embodiments, R₁ is selected from OH, CH₃O, NO₂, NH₂, and CN.

In some embodiments, R₁ is selected from OH, CH₃O, NO₂ and CF₃O.

In some embodiments, R₁ is selected from OH, CH₃O, NO₂ and NH₃⁺.

In some embodiments, R₁ is selected from OH, CH₃O, NO₂ and CF₃.

In some embodiments, R₁ is selected from OH, CH₃O, NO₂ and CN.

In some embodiments, R₁ is selected from OH, CH₃O, NO₂ and NH₂.

In some embodiments, R₁ is selected from OH, CH₃O, and NO₂.

In some embodiments, R₁ is selected from OH and CH₃O.

In some embodiments, R₁ is selected from CH₃O and NH₂.

In some embodiments, R₁ is selected from CH₃O and NO₂.

In some embodiments, R₁ is selected from OH and NO₂.

In some embodiments, R₁ is selected from OH and NH₂.

In some embodiments, R₁ is selected from OH and CN.

In some embodiments, R₁ is selected from OH and CF₃.

In some embodiments, R₁ is selected from OH and CF₃O.

In some embodiments, R₁ is selected from CH₃O and NO₂.

In some embodiments, R₁ is selected from CH₃O and CF₃O.

In some embodiments, R₁ is selected from CH₃O and CN.

In some embodiments, R₁ is selected from CH₃O and CF₃.

In some embodiments, R₁ is selected from CF₃O and CF₃.

In some embodiments, R₁ is selected from NH₂ and NH₃⁺.

In some embodiments, R₁ is selected from NH₂ and CN.

In some embodiments, R₁ is OH.

In some embodiments, R₁ is CH₃O.

In some embodiments, R₁ is NH₂.

In some particular embodiments, R₁ is NO₂, i.e. the compound of formula (I) may be represented by formula (Ib)

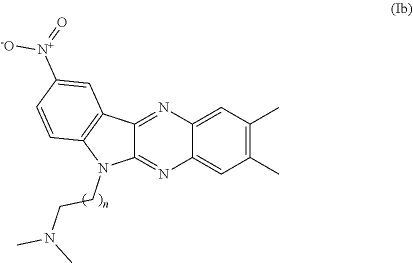

wherein n is 1 or 2.

In some embodiments of a compound of formula (Ib), n is 2. In some other embodiments, n is 1, i.e. the compound of formula (Ib) is is 2-(2,3-dimethyl-9-nitro-6H-indolo[2,3-b]quinoxalin-6-yl)-N,N-dimethylethanamine (which may also be referred to as 2-(2,3-dimethyl-9-nitro-6H-indolo[2,3-b]quinoxalin-6-yl)-N,N-dimethylethanamine), represented by formula

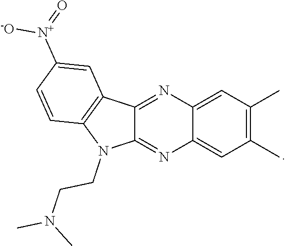

Some of the compounds of formula (I) as defined herein have not previously been proposed for use in therapy. Therefore, a further aspect is a compound of formula (I), or a pharmaceutically acceptable salt thereof, for use in therapy, wherein n is 1 or 2, e.g. n is 1; and R₁ is selected from NO₂, NH₂, CN, NH₃⁺, and CF₃O, or from any of the subgroups thereof, as mentioned herein above, for example, from NO₂, NH₂, CN, and NH₃⁺; or from NO₂, NH₂, and CN; or from NO₂, NH₂, and NH₃⁺; or from NO₂ and CN; or R₁ is NO₂; or R₁ is NH₂ or NH₃₄; or R₁ is NH₂; or R₁ is CN; or R₁ is CF₃O.

Some of the compounds of formula (I) as defined herein above are novel compounds. Thus, in some embodiments, a novel compound of formula (I) or a pharmaceutically acceptable salt thereof is provided, wherein n is 1 or 2, and R₁ is CN, i.e. a compound of formula (Ic)

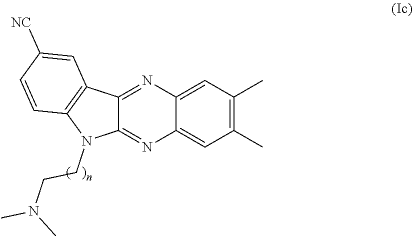

or a pharmaceutically acceptable salt thereof, wherein n is 1 or 2.

In some embodiments of a compound of formula (Ic), n is 1, i.e. the compound is 6-(2-(dimethylamino)ethyl)-2,3-dimethyl-6H-indolo[2,3-b]quinoxaline-9-carbonitrile.

Methods of Preparation

A compound of formula (I) may be prepared by following the method as described herein, or e.g. by methods as generally described in WO 87/04436 and in WO 96/19996, e.g. in Examples 1, 3 and 4 of WO 96/19996, the contents of which are incorporated herein by reference. Methods for preparing certain compounds of formula (I), in particular 6-(2-(dimethylamino)ethyl)-2,3-dimethyl-6H-indolo[2,3-b]quinoxalin-9-ol and 2,3-dimethyl-6-(N,N-dimethylaminoethyl)-9-methoxy-6H-indolo-(2,3-b)quinoxaline (JBA-094), also are described in a doctoral thesis titled “Syntheses of some tri- and tetracyclic heterocycles containing an indole moiety” (ISBN: 91-7140-161-X) (2004), the contents of which is incorporated herein cf. e.g. pages 40-42.

A compound of the general formula (Ic) may be prepared, for example, by following the method described in Example 1, using the appropriate 6H-indolo[2,3-b]quinoxaline derivative as a starting material.

Finally, it will be realized that there is a close link between a compound of formula (I) wherein R₁ is NH₂, and a compound of formula (I) wherein R₁ is NH₃⁺. Thus, the latter may be obtained as an acid addition salt of the former, e.g. by reacting the former with an appropriate acid, e.g. a hydrohalic acid, such as hydrochloric acid.

Pharmaceutically Acceptable Salts

A pharmaceutically acceptable salt of the compound of formula (I) may be an acid addition salt or a base addition salt.

In the preparation of acid or base addition salts, such acids or bases are used which form suitable pharmaceutically acceptable salts. Examples of such acids are inorganic acids such as hydrohalic acids, such as hydrochloric acid, sulfuric acid, phosphoric acid, nitric acid; organic aliphatic, alicyclic, aromatic or heterocyclic carboxylic or sulfonic acids, such as formic acid, acetic acid, propionic acid, succinic acid, glycolic acid, lactic acid, malic acid, tartaric acid, citric acid, ascorbic acid, maleic acid, hydroxymaleic acid, pyruvic acid, p-hydroxybenzoic acid, embonic acid, methanesulfonic acid, ethanesulfonic acid, hydroxyethanesulfonic acid, halogenbenzenesulfonic acid, toluenesulfonic acid or naphthalenesulfonic acid.

Base addition salts include those derived from inorganic bases, such as ammonium or alkali or alkaline earth metal hydroxides, carbonates, bicarbonates, and the like, and organic bases such as alkoxides, alkyl amides, alkyl and aryl amines, and the like. Examples of bases useful in preparing salts of the present invention include sodium hydroxide, potassium hydroxide, ammonium hydroxide, potassium carbonate, and the like.

Pharmaceutical Formulations

A pharmaceutical composition according to the invention may be for topical (local) or systemic administration, e.g. for enteral administration, such as rectal or oral administration, or for parenteral administration to a mammal (especially a human), and comprises a therapeutically effective amount of a compound according to the invention or a pharmaceutically acceptable salt thereof, as active ingredient, in association with a pharmaceutically acceptable excipient, e.g. a pharmaceutically acceptable carrier. The therapeutically effective amount of the active ingredient is as defined herein above and depends e.g. on the species of mammal, the body weight, the age, the individual condition, individual pharmacokinetic data, the disease to be treated and the mode of administration.

For enteral, e.g. oral, administration, the compounds of the invention may be formulated in a wide variety of dosage forms. The pharmaceutical compositions and dosage forms may comprise a compound or compounds of the present invention or pharmaceutically acceptable salt(s) thereof as the active component. The pharmaceutically acceptable carriers may be either solid or liquid. Solid form preparations include powders, tablets, pills, lozenges, capsules, cachets, suppositories, and dispersible granules. A solid carrier may be one or more substances which may also act as diluents, flavouring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. The formulation of the active compound may comprise an encapsulating material as carrier, providing a capsule in which the active component, with or without carriers, is surrounded by a carrier, which is in association with it.

Other forms suitable for oral administration include liquid form preparations including emulsions, syrups, elixirs, aqueous solutions, aqueous suspensions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

Exemplary compositions for rectal administration include suppositories which can contain, for example, a suitable non-irritating excipient, such as cocoa butter, synthetic glyceride esters or polyethylene glycols, which are solid at ordinary temperatures, but liquefy and/or dissolve in the rectal cavity to release the drug.

The compounds of the invention also may be administered parenterally, e.g. by inhalation, injection or infusion, e.g. by intravenous, intraarterial, intraosseous, intramuscular, intracerebral, intracerebroventricular, intrasynovial, intrasternal, intrathecal, intralesional, intracranial, intratumoral, intracutaneous and subcutaneous injection or infusion.

Thus, for parenteral administration, the pharmaceutical compositions of the invention may be in the form of a sterile injectable or infusible preparation, for example, as a sterile aqueous or oleaginous suspension. This suspension may be formulated according to techniques known in the art using suitable dispersing or wetting agents (e.g. Tween 80), and suspending agents. The sterile injectable or infusible preparation may also be a sterile injectable or infusible solution or suspension in a non-toxic parenterally acceptable diluent or solvent. For example, the pharmaceutical composition may be a solution in 1,3-butanediol. Other examples of acceptable vehicles and solvents that may be employed in the compositions of the present invention include, but are not limited to, mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant.

Solutions for parenteral use also may contain suitable stabilizing agents, and if necessary, buffer substances. Suitable stabilizing agents include antioxidizing agents, such as sodium bisulfate, sodium sulfite or ascorbic acid, either alone or combined, citric acid and its salts and sodium EDTA. Parenteral solutions may also contain preservatives, such as benzalkonium chloride, methyl- or propyl-paraben, and chlorobutanol.

For inhalation or nasal administration, suitable pharmaceutical formulations are as particles, aerosols, powders, mists or droplets, e.g. with an average size of about 10 μm in diameter or less. For example, compositions for inhalation may be prepared as solutions in saline, employing benzyl alcohol or other suitable preservatives, absorption promoters to enhance bioavailability, fluorocarbons, and/or other solubilizing or dispersing agents known in the art.

The pharmaceutical compositions of the invention also may be administered topically, to the skin or to a mucous membrane. For topical application, the pharmaceutical composition may be e.g. a lotion, a gel, a paste, a tincture, a transdermal patch, a gel for transmucosal delivery. The composition may be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition may be formulated as a suitable lotion or cream containing the active compound suspended or dissolved in a carrier. Suitable carriers include, but are not limited to, mineral oil, sorbitan monostearate, polysorbate 60, cetyl esters wax, cetaryl alcohol, 2-octyldodecanol, benzyl alcohol and water. The pharmaceutical compositions of this invention may also be topically applied to the lower intestinal tract by rectal suppository formulation or in a suitable enema formulation.

Suitable pharmaceutical excipients, e.g. carriers, and methods of preparing pharmaceutical dosage forms are described in Remington's Pharmaceutical Sciences, Mack Publishing Company, a standard reference text in art of drug formulation.

The pharmaceutical compositions may comprise from approximately 1% to approximately 95%, preferably from approximately 20% to approximately 90% of a compound of formula (I), together with at least one pharmaceutically acceptable excipient. In general, the compounds of the invention will be administered in a therapeutically effective amount by any of the accepted modes of administration for agents that serve similar utilities. Suitable daily dosages typically range from 1 to 1000 mg, e.g. 1-500 mg daily, or 1-50 mg daily, depending upon numerous factors such as the severity of the disease to be treated, the age and relative health of the patient, the potency of the compound used, the route and form of administration, and the indication towards which the administration is directed, etc. One of ordinary skill in the art of treating such diseases will be able, without undue experimentation and in reliance upon personal knowledge and the disclosure of this application, to ascertain a therapeutically effective amount of the compounds of the present invention for a given disease. Compounds of the invention may be administered as pharmaceutical formulations including those suitable for enteral or parenteral administration. The preferred manner of administration is generally oral using a convenient daily dosage regimen which can be adjusted according to the degree of affliction.

The compounds of the present invention may also be used or administered in combination with one or more additional therapeutically active agents, e.g. one or more further anticancer agents. The components may be in the same formulation or in separate formulations for administration simultaneously or sequentially.

In a further aspect of the invention, there is provided a combination product comprising:
(A) a compound of the invention, as defined herein; and
(B) another therapeutic agent, e.g. one that is useful in the treatment of a cancer; whereby (A) and (B) is formulated in admixture with a pharmaceutically acceptable excipient.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:
(1) a pharmaceutical formulation including a compound of the invention, as hereinbefore defined, another therapeutic agent, and a pharmaceutically acceptable excipient, e.g. an adjuvant, diluent or carrier; and/or
(2) a kit of parts comprising, as components:
(a) a pharmaceutical formulation including a compound of the invention, as defined herein, in admixture with a pharmaceutically acceptable excipient, e.g. an adjuvant, diluent or carrier; and
(b) a pharmaceutical formulation including another therapeutic agent in admixture with a pharmaceutically acceptable excipient, e.g. an adjuvant, diluent or carrier, which components (a) and (b) are each provided in a form that is suitable for administration in conjunction with the other.

The compounds of the present invention may also be used or administered in combination with other treatment such as irradiation for the treatment of cancer.

A further aspect is a process for the preparation of a pharmaceutical formulation, which process suitably comprises bringing into association a compound of the invention, as herein defined, or a pharmaceutically acceptable salt thereof, with a pharmaceutically acceptable excipient, e.g. an adjuvant, diluent or carrier.

A further aspect is the use of a compound of formula (I) as defined herein, for the manufacture of a medicament for the treatment of cancer.

A further aspect is a method of treating cancer comprising administering to a patient in need thereof a therapeutically effective amount of a compound of formula (I), as herein defined.

Methods of Treatment

The compounds of the invention may be administered to a subject in need of treatment e.g. by use of a pharmaceutical formulation and administration route as generally outlined herein above, however it should be realized that precise treatment regime, e.g. dosage, will normally be determined by the treating physician.

The skilled person will understand that references to the treatment of a particular cancer (or, similarly, to treating that condition) take their normal meanings in the field of medicine. In particular, the terms may refer to achieving a reduction in the severity of one or more clinical symptom associated with the cancer. For example, the term may refer to achieving a reduction of the amount (i.e. the number) of cancerous cells present (which may, in the case of a cancer forming a solid tumour, be indicated by a reduction in tumour volume).

As used herein, references to patients will refer to a living subject being treated, including mammalian (e.g. human) patients.

As used herein, the term effective amount will refer to an amount of a compound that confers a therapeutic effect on the treated patient. The effect may be observed in a manner that is objective (i.e. measurable by some test or marker) or subjective (i.e. the subject gives an indication of and/or feels an effect).

The skilled person will understand that parameters such as number of tumour cells present and/or the volume of a tumour (and, consequently, the reduction thereof as measured between two or more time points) may be observed and measured using techniques well-known to those skilled in the art, such as by using scanning techniques (e.g. MRI scan) and/or the taking and analysis of samples (such as blood samples).

In some embodiments, the treatment may also include surgery and/or irradiation therapy.

The Cancer

The cancer that may be treated by administration of a compound of the present invention is as defined and mentioned herein. In some embodiments, the cancer is a malignant solid tumor.

In some embodiments, the cancer is a hematological cancer (e.g. leukemia), a lung cancer (e.g. non-small cell lung cancer), a colon cancer, a cancer of the CNS, a skin cancer (e.g. malignant melanoma), an ovarian cancer, a renal cancer, a prostate cancer or a breast cancer.

In some embodiments, the cancer is a lung cancer (e.g. non-small cell lung cancer), a colon cancer, a cancer of the CNS, a skin cancer (e.g. malignant melanoma), an ovarian cancer, a renal cancer, a prostate cancer or a breast cancer.

In some embodiments, when the cancer is a hematological cancer, the cancer is not lymphoma. In some embodiments, when the cancer is a hematological cancer, the cancer is leukemia.

In some embodiments, the cancer is a hematological cancer, e.g. the cancer is leukemia.

In some further embodiments, the cancer is a malignant solid tumor, e.g. colon cancer, cancer of the CNS, skin cancer (e.g. malignant melanoma), ovarian cancer, renal cancer, prostate cancer, or breast cancer.

In some embodiments, the cancer is colon cancer. In some embodiments, the cancer is a cancer of the CNS, e.g. glioblastoma. In some embodiments, the cancer is skin cancer (e.g. malignant melanoma). In some embodiments, the cancer is ovarian cancer. In some embodiments, the cancer is renal cancer. In some embodiments, the cancer is prostate cancer. In some embodiments, the cancer is breast cancer.

The invention is further illustrated by the following non-limitative examples.

### EXAMPLES

General Procedures

Analytical HPLC-MS was performed using an Agilent 1100 series Liquid

Chromatograph/Mass Selective Detector (MSD) (Single Quadrupole) equipped with an electrospray interface and a UV diode array detector. Analyses were performed by two methods using either an ACE 3 C8 (3.0×50 mm) column with a gradient of acetonitrile in 0.1% aqueous TFA over 3 min and a flow of 1 mL/min, or an XBridge C18 (3.0×50 mm) column with a gradient of acetonitrile in 10 mM ammonium bicarbonate over 3 min and a flow of 1 mL/min. NMR spectra were recorded on a Bruker 400 MHz instrument at 25° C.

### Example 1

### 6-(2-(dimethylamino)ethyl)-2,3-dimethyl-6H-indolo[2,3-b]quinoxaline-9-carbonitrile

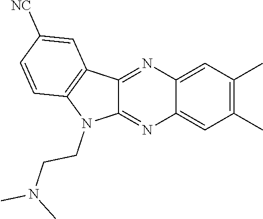

2-(9-bromo-2,3-dimethyl-6H-indolo[2,3-b]quinoxalin-6-yl)-N,N-dimethylethanamine (68 mg, 0.17 mmol), Zn(CN)₂ (23 mg, 0.20 mmol, 1.1 eq), and tetrakis(triphenylphosphine) palladium(0) (68 mg, 0.058 mmol, 0.3 eq), were weighed in a microwave vial. To the solids was added DMF (1 mL) and nitrogen was bubbled through for 5 minutes. The mixture was then heated to 130° C. in an oil bath and stirred for 18 hours. Upon completion the reaction mixture was added to 1 mL water and 1 mL Et₂O. The Et₂O layer was taken up and the aqueous phase was extracted with Et₂O twice more. The combined organic layers were dried over MgSO₄, filtered (this also removed some Pd-residues, on a larger scale this should be filtered off first), and concentrated under vacuum. The yellow solid crude (containing some DMF) was purified using preparative HPLC (basic method, 30-60% acetonitrile), to obtain 35 mg (>97% pure, 60% yield) after freeze drying.

MS (ESI+) m/z 344 [M+H]+.

1H NMR (CDCl₃, ppm) 2.39 (s, 6H), 2.55-2.60 (m, 6H), 2.86 (t, 2H), 4.64 (t, 2H), 7.60 (d, 1H), 7.91 (d, 1H), 7.93 (s, 1H), 8.08 (s, 1H), 8.76 (s, 1H).

### Example 2

Human tumor cell lines were grown in RPMI 1640 medium containing 5% fetal bovine serum and 2 mM L-glutamine. Cells were inoculated into 96 well microtiter plates in 100 μL at plating densities ranging from 5,000-40,000 cells/well. After cell inoculation, the plates were incubated at 37° C., 5% CO₂, 95% air and 100% relative humidity for 24 h prior to addition of tested compounds.

After 24 h, two plates of each cell line were fixed in situ with TCA, to represent a measurement of the cell population for each cell line at the time of drug addition (Tz). Tested compounds were solubilized in DMSO at 400-fold the desired final maximum test concentration and stored frozen prior to use. At the time of compound addition, an aliquot of frozen concentrate was thawed and diluted to twice the desired final maximum test concentration with complete medium containing 50 μg/ml gentamicin. Additional four, 10-fold or ½ log serial dilutions were made to provide a total of five drug concentrations plus control. Aliquots of 100 μl of these different compound dilutions were added to the appropriate microtiter wells already containing 100 μl of medium, resulting in the required final concentrations.

Following addition of the tested compounds, the plates were incubated for an additional 48 h at 37° C., 5% CO₂, 95% air, and 100% relative humidity. For adherent cells, the assay was terminated by the addition of cold TCA. Cells were fixed in situ by the gentle addition of 50 μl of cold 50% (w/v) TCA (final concentration, 10% TCA) and incubated for 60 min at 4° C. The supernatant was discarded, and the plates were washed five times with tap water and air dried. Sulforhodamine B (SRB) solution (100 μl) at 0.4% (w/v) in 1% acetic acid was added to each well, and plates were incubated for 10 min at room temperature. After staining, unbound dye was removed by washing five times with 1% acetic acid and the plates were air dried. Bound stain was subsequently solubilized with 10 mM trizma base, and the absorbance was read on a plate reader at 515 nm. For suspension cells, the methodology was the same except that the assay was terminated by fixing settled cells at the bottom of the wells by gently adding 50 μl of 80% TCA (final concentration, 16% TCA). Using the seven absorbance measurements [time zero, (Tz), control growth, (C), and test growth in the presence of tested compound at the five concentration levels (Ti)], the percentage growth was calculated at each of the concentration levels. Percentage growth inhibition was calculated as:

[(Ti−Tz)/(C−Tz)]×100 for concentrations for which Ti>/=Tz

[(Ti−Tz)/Tz]×100 for concentrations for which Ti<Tz.

Three dose response parameters were calculated for each tested compound. Growth inhibition of 50% (GI₅₀) was calculated from [(Ti−Tz)/(C−Tz)]×100=50, which is the concentration of compound resulting in a 50% reduction in the net protein increase (as measured by SRB staining) in control cells during the incubation with compound. The concentration of compound that resulted in total growth inhibition (TGI) was calculated from Ti=Tz. The LC₅₀ (concentration of compound resulting in a 50% reduction in the measured protein at the end of the drug treatment as compared to that at the beginning) indicating a net loss of cells following treatment was calculated from [(Ti−Tz)/Tz]×100=−50. Values were calculated for each of these three parameters if the level of activity was reached; however, if the effect was not reached or was exceeded, the value for that parameter is expressed as greater or less than the maximum or minimum concentration tested. Tested compounds are shown in TABLE 1.

TABLE 1

| Name | Chemical name | Structural formula |
|---|---|---|
| B220* | 2-(2,3-dimethyl-6H-indolo [2,3-b]quinoxalin-6-yl)- N,N-dimethylethanamine | 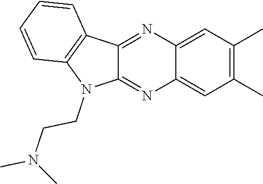 |
| JBA-185 | 2-(2,3-dimethyl-9-nitro-6H- indolo[2,3-b]quinoxalin-6-yl)- N,N-dimethylethanamine | 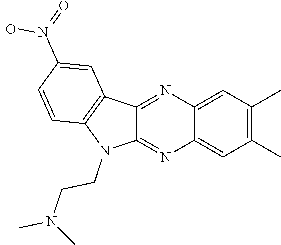 |
| JBD-136 | 6-(2-(dimethylamino)ethyl)- 2,3-dimethyl-6H-indolo [2,3-b]quinoxalin-9-amine | 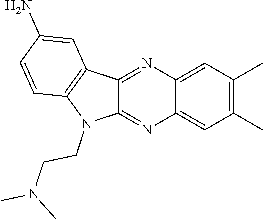 |
| JBD-139 | 6-(2-(dimethylamino)ethyl)- 2,3-dimethyl-6H-indolo [2,3-b]quinoxalin-9-ol | 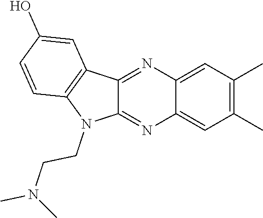 |
| JBA-094 | 2-(9-methoxy-2,3-dimethyl- 6H-indolo[2,3-b]quinoxalin- 6-yl)-N,N- dimethylethanamine | 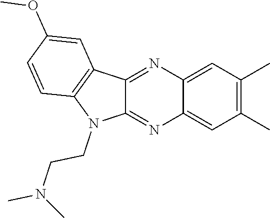 |

*Not according to the invention

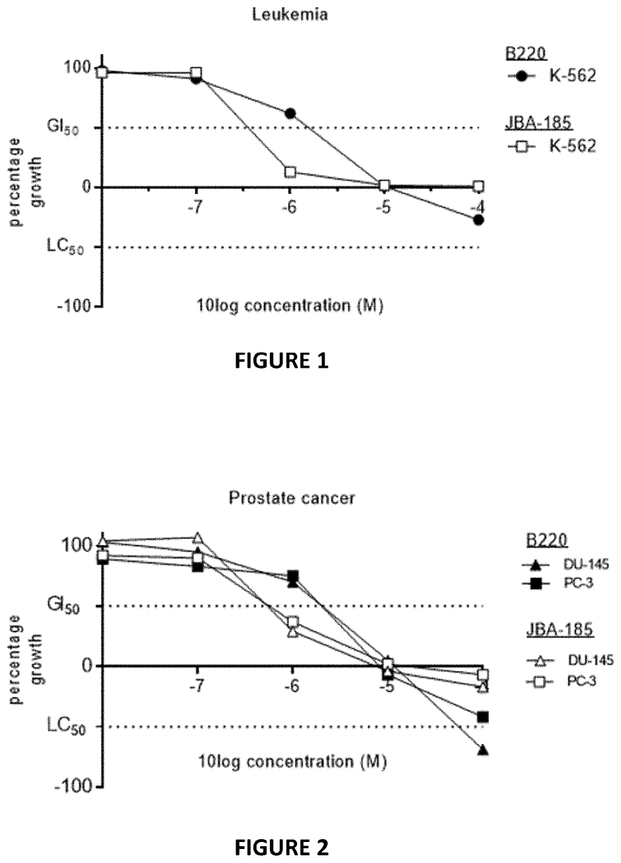

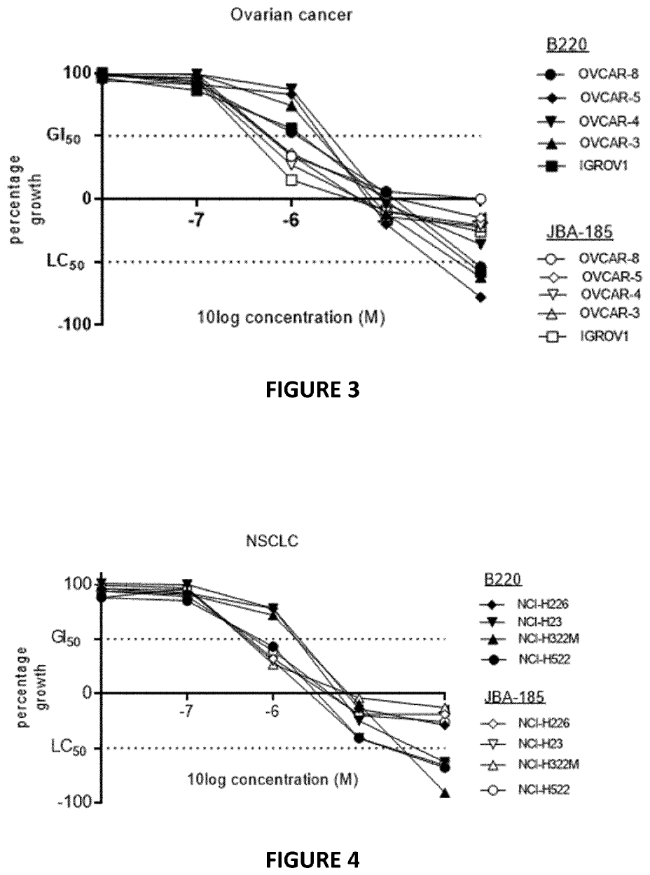

Results, in terms of lethality (LC₅₀) and growth inhibition (GI₅₀), for JBA-185, JBD-136, JBD-139, JBA-094, and for compound B220 (not according to the invention), are presented in TABLE 2 and TABLE 3, respectively. Some of the results obtained are additionally illustrated in FIGS. 1-4.

TABLE 2

| | | | | | |
|---|---|---|---|---|---|
| | JBA-185 | JBD-136 | JBD-139 | JBA-094 | B220 |
| Cancer Cell | LC₅₀ | LC₅₀ | LC₅₀ | LC₅₀ | LC₅₀ |
| Line | μM | μM | μM | μM | μM |

Leukemia

| | | | | | |
|---|---|---|---|---|---|
| CCRF-CEM | >10² | 76.9 | >10² | >10² | >10² |
| HL-60(TB) | >10² | 21.9 | — | >10² | >10² |
| K-562 | >10² | 65.6 | >10² | >10² | >10² |
| MOLT-4 | >10² | 71.9 | >10² | 96.0 | 95.1 |
| RPMI-8226 | >10² | 95.2 | — | — | — |
| SR | >10² | >10² | >10² | >10² | — |

NSCLC

| | | | | | |
|---|---|---|---|---|---|
| A549/ATCC | 94.4 | >10² | — | 7.97 | >10² |
| EKVX | >10² | >10² | — | 6.8 | 83.9 |
| HOP-62 | >10² | >10² | >10² | >10² | >10² |
| HOP-92 | >10² | >10² | >10² | 6.08 | >10² |
| NCI-H226 | >10² | >10² | 19.5 | 8.2 | >10² |
| NCI-H23 | 22.6 | 33 | — | 8.2 | 45.6 |
| NCI-H322M | >10² | >10² | >10² | 6.41 | 31.2 |
| NCI-H460 | 26.7 | 36.6 | — | 4.98 | 37.3 |
| NCI-H522 | >10² | >10² | >10² | >10² | >10² |

Colon

| | | | | | |
|---|---|---|---|---|---|
| COLO 205 | >10² | 83.6 | >10² | — | 41.0 |
| HCC-2998 | 6.53 | 21.9 | — | 6.32 | 15.7 |
| HCT-116 | 94.2 | 51.4 | — | 6.59 | 44.5 |
| HCT-15 | >10² | >10² | >10² | 5.57 | >10² |
| HT29 | >10² | >10² | >10² | 5.72 | 45.3 |
| KM12 | >10² | 94.7 | — | 6.36 | 38.6 |
| SW-620 | >10² | 83.8 | — | 5.53 | 35.0 |

CNS

| | | | | | |
|---|---|---|---|---|---|
| SF-268 | >10² | 42.7 | >10² | 8.23 | 28.0 |
| SF-295 | 8.64 | 33.8 | >10² | 5.13 | 42.5 |
| SF-539 | 18.1 | 19.5 | — | 5.2 | 15.0 |
| SNB-19 | >10² | >10² | >10² | 6.34 | 68.6 |
| SNB-75 | 5.45 | 24.8 | >10² | 23.8 | 80.2 |
| U251 | >10² | 54.2 | — | 6.09 | 44.6 |

Melanoma

| | | | | | |
|---|---|---|---|---|---|
| LOX IMVI | | 21.8 | — | 6.07 | 18.9 |
| MALME-3M | 5.10 | 7.43 | — | 5.77 | 53.3 |
| M14 | 7.28 | 6.43 | 5.15 | 5.89 | 75.1 |
| MDA-MB-435 | 4.50 | 5.6 | — | 6.03 | 7.49 |
| SK-MEL-2 | 9.02 | 66.7 | — | 7.87 | 7.49 |
| SK-MEL-28 | 5.65 | 11.4 | — | 6.76 | 9.45 |
| SK-MEL-5 | 4.77 | 5.72 | 5.33 | 5.58 | 12.0 |
| UACC-257 | 34.4 | 71.5 | — | 7.97 | 5.99 |
| UACC-62 | 5.78 | 7.99 | 6.37 | 6.7 | 5.58 |

Ovarian

| | | | | | |
|---|---|---|---|---|---|
| IGR-OV1 | >10² | >10² | 98.2 | 5.41 | 72.4 |
| OVCAR-3 | >10² | 75.8 | — | 6.13 | 56.8 |
| OVCAR-4 | >10² | 59 | — | 5.56 | >10² |
| OVCAR-5 | >10² | >10² | >10² | 5.28 | 33.4 |
| OVCAR-8 | >10² | >10² | >10² | — | 87.2 |
| NCI/ADR-RES | >10² | >10² | >10² | — | 90.2 |
| SK-OV-3 | >10² | >10² | >10² | 7.17 | >10² |

Renal

| | | | | | |
|---|---|---|---|---|---|
| 786-0 | 9.56 | 38.5 | — | 6.52 | 62.0 |
| A498 | 5.50 | 5.86 | 5.69 | 5.56 | 5.74 |
| ACHN | 2.46 | 57.3 | — | 3.96 | 42.2 |
| CAKI-1 | 7.48 | 85.5 | 5.31 | 4.93 | >10² |
| RXF 393 | 5.50 | 6.4 | 5.96 | 6.9 | 12.2 |
| SN12C | >10² | >10² | >10² | 5.24 | >10² |
| TK-10 | >10² | >10² | >10² | 6.92 | 71.4 |
| UO-31 | 21.2 | 55.3 | 4.18 | 4.93 | 91.3 |

Prostate

| | | | | | |
|---|---|---|---|---|---|
| PC-3 | >10² | 51.9 | — | 5.84 | >10² |
| DU-145 | >10² | 47.8 | >10² | 5.32 | 55.6 |

Breast

| | | | | | |
|---|---|---|---|---|---|
| MCF7 | >10² | 57.5 | >10² | 4.79 | 34.8 |
| MDA-MB-231/ | >10² | 57.5 | — | 6.39 | 54.9 |
| ATCC | | | | | |
| MDA-MB-468 | 5.30 | 8.28 | >10² | 6.9 | 9.93 |
| HS 578T | >10² | >10² | >10² | 78.7 | 67.4 |
| BT-549 | >10² | 27.9 | >10² | 7.09 | 28.7 |
| T-47D | >10² | >10² | — | >10² | >10² |

TABLE 3

| | | | | | |
|---|---|---|---|---|---|
| | JBA-185 | JBD-136 | JBD-139 | JBA-094 | B220 |
| Cancer Cell | GI₅₀ | GI₅₀ | GI₅₀ | GI₅₀ | GI₅₀ |
| Line | μM | μM | μM | μM | μM |

Leukemia

| | | | | | |
|---|---|---|---|---|---|
| CCRF-CEM | 0.642 | 0.862 | 0.468 | 0.795 | 1.99 |
| HL-60(TB) | 0.914 | 1.21 | 0.356 | 1.18 | 1.99 |
| K-562 | 0.360 | 0.426 | 0.338 | 0.444 | 1.99 |
| MOLT-4 | 0.368 | 0.366 | 0.359 | 0.42 | 0.555 |
| RPMI-8226 | 0.389 | 0.74 | 0.366 | 0.64 | — |
| SR | 0.250 | 0.239 | 0.289 | 0.409 | — |

NSCLC

| | | | | | |
|---|---|---|---|---|---|
| A549/ATCC | 0.387 | 0.464 | 0.358 | 0.482 | 1.28 |
| EKVX | 0.477 | 1.99 | 1.1 | 1.28 | 2.23 |
| HOP-62 | 0.507 | 1.14 | 0.569 | 1.19 | 1.11 |
| HOP-92 | 0.642 | 0.912 | 0.246 | 0.238 | 1.36 |
| NCI-H226 | 0.521 | 1.32 | 1.34 | 1.24 | 2.01 |
| NCI-H23 | 0.492 | 1.08 | 0.506 | 0.973 | 1.87 |
| NCI-H322M | 0.467 | 0.771 | 0.516 | 0.778 | 1.86 |
| NCI-H460 | 0.350 | 0.434 | 0.348 | 0.388 | 0.709 |
| NCI-H522 | 0.595 | 0.771 | 0.39 | 0.515 | 0.673 |

Colon

| | | | | | |
|---|---|---|---|---|---|
| COLO 205 | 0.360 | 0.418 | 0.47 | 0.561 | 1.29 |
| HCC-2998 | 0.756 | 1.02 | 0.75 | 1.08 | 1.47 |
| HCT-116 | 0.355 | 0.369 | 0.326 | 0.312 | 0.488 |
| HCT-15 | 0.349 | 0.528 | 0.405 | 0.41 | 1.77 |
| HT29 | 0.375 | 0.43 | 0.379 | 0.369 | 0.760 |
| KM12 | 0.567 | 1.2 | 0.512 | 1.05 | 1.93 |
| SW-620 | 0.302 | 0.389 | 0.32 | 0.454 | 0.540 |

CNS

| | | | | | |
|---|---|---|---|---|---|
| SF-268 | 0.793 | 1.57 | 0.822 | 1.33 | 1.45 |
| SF-295 | 0.388 | 1.24 | 0.756 | 1.11 | 1.39 |
| SF-539 | 0.387 | 0.882 | 0.422 | 0.663 | 1.83 |
| SNB-19 | 0.493 | 0.767 | 0.423 | 0.719 | 2.33 |
| SNB-75 | 0.447 | 1.25 | 0.214 | 1.23 | 1.28 |
| U251 | 0.470 | 0.64 | 0.356 | 0.526 | 2.01 |

Melanoma

| | | | | | |
|---|---|---|---|---|---|
| LOX IMVI | | 0.494 | 0.338 | 0.336 | 0.748 |
| MALME-3M | 0.542 | 1.27 | 0.623 | 1.11 | 1.41 |
| M14 | 0.499 | 1.12 | 0.378 | 1.12 | 1.51 |
| MDA-MB-435 | 0.541 | 1.05 | 0.529 | 1.11 | 1.61 |
| SK-MEL-2 | 1.04 | 1.63 | 1.1 | 1.71 | 1.90 |
| SK-MEL-28 | 0.807 | 2.26 | 1.5 | 1.79 | 2.09 |
| SK-MEL-5 | 1.04 | 1.69 | 1.45 | 1.66 | 1.70 |
| UACC-257 | 0.732 | 1.5 | 0.597 | 1.5 | 1.94 |
| UACC-62 | 0.514 | 1.18 | 0.479 | 0.898 | 1.43 |

Ovarian

| | | | | | |
|---|---|---|---|---|---|
| IGR-OV1 | 0.357 | 0.596 | 0.392 | 0.396 | 1.28 |
| OVCAR-3 | 0.590 | 1.56 | 0.97 | 1.26 | 1.91 |
| OVCAR-4 | 0.452 | 1.7 | 0.882 | 1.02 | 2.55 |
| OVCAR-5 | 0.587 | 1.24 | 0.867 | 1.12 | 2.10 |
| OVCAR-8 | 0.550 | 0.556 | 0.412 | 0.531 | 1.16 |
| NCI/ADR-RES | 0.415 | 0.506 | 0.436 | 0.588 | 1.39 |
| SK-OV-3 | 0.704 | 2.23 | 1.16 | 1.5 | 3.10 |

Renal

| | | | | | |
|---|---|---|---|---|---|
| 786-0 | 0.757 | 1.52 | 0.707 | 1.04 | 1.97 |
| A498 | 1.26 | 1.07 | 0.948 | 1.48 | 1.43 |
| ACHN | 0.316 | 0.367 | 0.307 | 0.358 | 0.555 |
| CAKI-1 | 0.251 | 0.427 | 0.259 | 0.541 | 0.982 |
| RXF 393 | 0.638 | 1.13 | 0.671 | 1.18 | 1.56 |
| SN12C | 0.479 | 0.553 | 0.397 | 0.503 | 1.08 |
| TK-10 | 0.371 | 2.26 | 1.74 | 1.96 | 2.97 |
| UO-31 | 0.187 | 0.332 | 1.39 | 0.273 | 0.726 |

Prostate

| | | | | | |
|---|---|---|---|---|---|
| PC-3 | 0.187 | 1.28 | 0.491 | 1.12 | 2.01 |
| DU-145 | 0.540 | 1.55 | 0.598 | 1.01 | 2.07 |

Breast

| | | | | | |
|---|---|---|---|---|---|
| MCF7 | 0.356 | 0.675 | 0.351 | 0.332 | 1.41 |
| MDA-MB-231/ | 0.495 | 1.22 | 0.544 | 1.14 | 1.76 |
| ATCC | | | | | |
| MDA-MB-468 | 0.374 | 1.03 | 0.518 | 0.749 | 1.32 |
| HS 578T | 1.57 | 2.12 | 1.4 | 1.93 | 1.57 |
| BT-549 | 1.95 | 1.78 | 1.84 | 1.65 | 2.07 |
| T-47D | 0.499 | 2.26 | 0.686 | 1.32 | 2.80 |

The results show that JBA-185 is less cytotoxic than B220, having an LC₅₀>10² μM for most cell lines. JBA-185 further generally has a better ability to inhibit cancer cell growth as shown by the lower GI₅₀ compared to B220. The results are indicative of an advantageously larger selectivity index of JBA-185, compared to B220. The results for JBD-136 and JBD-139 are overall better compared to B220, but generally not as good as for JBA-185. JBD-139 demonstrates similar growth inhibition (GI₅₀) as JBA-185, but with a cytotoxicity that is higher. JBD-136 demonstrates a similar however slightly improved growth inhibition and cytotoxicity compared to B220.

1.-16. (canceled)

17. A compound of formula (Ic), 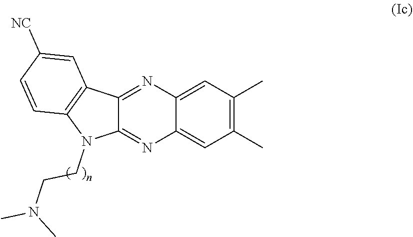
or a pharmaceutically acceptable salt thereof,
wherein n is 1 or 2.

18. The compound of claim 17, wherein n is 2.

19. A pharmaceutical composition comprising a compound of formula (I) 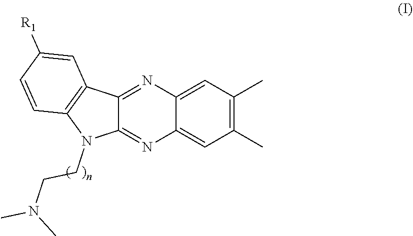
or a pharmaceutically acceptable salt thereof, wherein
n is 1 or 2; and
R₁ is selected from NO₂, NH₂, CN, NH₃⁺, and CF₃O;
and optionally a pharmaceutically acceptable excipient.

20. (canceled)

21. A method for the treatment of a cancer selected from a hematological cancer, lung cancer, colon cancer, cancer of the CNS, skin cancer, ovarian cancer, renal cancer, prostate cancer and breast cancer in a mammal by administering, to a mammal in need of such treatment, an effective amount of a compound of formula (I) 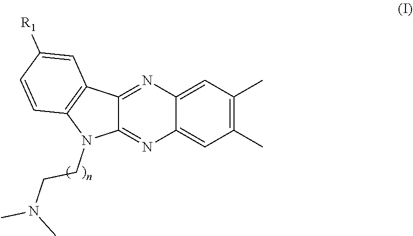
or a pharmaceutically acceptable salt thereof, wherein
n is 1 or 2; and
R₁ is selected from OH, NO₂, NH₂, CN, CF₃, NH₃⁺, CH₃O, and CF₃O.

22. The pharmaceutical composition according to claim 19, wherein R₁ is selected from NO₂, NH₂, and CN.

23. The pharmaceutical composition according to claim 19, wherein R₁ is selected from NO₂, and NH₂.

24. The pharmaceutical composition according to claim 19, wherein R₁ is NO₂.

25. The pharmaceutical composition according to claim 19, wherein n is 1.

26. The pharmaceutical composition according to claim 19, wherein the compound is selected from:
2-(2,3-dimethyl-9-nitro-6H-indolo[2,3-b]quinoxalin-6-yl)-N,N-dimethylethanamine,
6-(2-(dimethylamino)ethyl)-2,3-dimethyl-6H-indolo[2,3-b]quinoxalin-9-amine, and
6-(2-(dimethylamino)ethyl)-2,3-dimethyl-6H-indolo[2,3-b]quinoxaline-9-carbonitrile.

27. The pharmaceutical composition according to claim 26, wherein the compound is selected from:
2-(2,3-dimethyl-9-nitro-6H-indolo[2,3-b]quinoxalin-6-yl)-N,N-dimethylethanamine, and
6-(2-(dimethylamino)ethyl)-2,3-dimethyl-6H-indolo[2,3-b]quinoxalin-9-amine.

28. The pharmaceutical composition according to claim 19, wherein the compound is 2-(2,3-dimethyl-9-nitro-6H-indolo[2,3-b]quinoxalin-6-yl)-N,N-dimethylethanamine.

29. The method according to claim 21, wherein R₁ is selected from OH, NO₂, NH₂, CN, NH₃⁺, and CH₃O.

30. The method according to claim 21, wherein R₁ is selected from OH, NO₂, NH₂, and CN.

31. The method according to claim 21, wherein R₁ is selected from OH, NO₂ and NH₂.

32. The method according to claim 21, wherein n is 1.

33. The method according to claim 21, wherein the compound is selected from:
2-(2,3-dimethyl-9-nitro-6H-indolo[2,3-b]quinoxalin-6-yl)-N,N-dimethylethanamine,
6-(2-(dimethylamino)ethyl)-2,3-dimethyl-6H-indolo[2,3-b]quinoxalin-9-amine,
6-(2-(dimethylamino)ethyl)-2,3-dimethyl-6H-indolo[2,3-b]quinoxalin-9-ol,
2,3-dimethyl-6-(N,N-dimethylaminoethyl)-9-methoxy-6H-indolo-(2,3-b)quinoxaline, and
6-(2-(dimethylamino)ethyl)-2,3-dimethyl-6H-indolo[2,3-b]quinoxaline-9-carbonitrile.
